(19) United States
(12) Patent Application Publication
(10) Pub. No.: US 2017/0189633 A1
(43) Pub. Date: Jul. 6, 2017

(54) TRACHEAL TUBE WITH IMPROVED SUCTION CAPABILITY

(71) Applicant: Flosure Technologies LLC, Tarrytown, NY (US)

(72) Inventor: Gerald W. Gentile, Yonkers, NY (US)

(21) Appl. No.: 15/396,226

(22) Filed: Dec. 30, 2016

Related U.S. Application Data
(60) Provisional application No. 62/273,153, filed on Dec. 30, 2015.

(51) Int. Cl. A61M 16/04

(52) U.S. Cl. CPC A61M 16/0479; A61M 16/044; A61M 16/0463

(57) ABSTRACT

The invention provides a tracheal tube having at least two suction ports, located very closely to an inflatable cuff. The ports open out onto channels which together subtend an angle of almost 360° around the tube, providing efficient and thorough suctioning of fluids that may have accumulated in the subglottal region above the cuff.

### RELATED APPLICATIONS

This application claims priority of U.S. provisional Patent application No. 62/273,153 filed on Dec. 30, 2015, the contents of which are incorporated herein by reference in their entirety.

### FIELD OF THE INVENTION

The invention relates to a tracheal tubes, more specifically to a tracheal tube having a plurality of suction ports providing roughly 360° of suction for fluid removal.

### BACKGROUND OF THE INVENTION

Tracheotomy is a surgical incision directly into the anterior aspect of the trachea for the purpose of establishing an artificial airway. A tracheal tube is placed into the surgically created opening to maintain the airway, resulting in what is usually referred to as a tracheostomy. An inflatable cuff toward the distal end of the tube is used to create a seal, so that a mechanical ventilator can inflate and deflate the lungs via the tube. One potential danger associated with tracheostomies is that fluid may collect above the cuff, which may contribute to a host of infections or other conditions (e.g. ventilator-associated tracheobronchitis (VAT) and ventilator-associated pneumonia (VAP).

Clinicians conventionally suction out fluids that may have collected above the cuff using an electric suction pump, a wall-installed suction connection, or a manual syringe, via a lumen and port built into the tracheal tube for this purpose. However, tracheal tubes currently on the market have deficiencies which contribute to inadequate fluid removal. For instance, current devices typically provide a single suction port, for the task of suctioning the area 360° around the perimeter of the tracheostomy tube. When a patient is in a reclining or semi-reclining position, the suction port may not be positioned at the lowest point around the perimeter, and fluid collecting below the suction port will not be removed. Moreover, due to manufacturing constraints, the suction port provided on most tracheostomy tubes is situated well above the cuff, permitting fluid to accumulate above the cuff to a level as high as 0.5 inches before it reaches the suction port. This allows for unnecessary and potentially harmful pooling of fluids, and does not provide for optimal removal of the fluids.

There is therefore a need in the art for a tracheal tube that has the capability to suction a 360° area around the tube, and which provides a suctioning port close to the cuff, to enable effective removal of fluids that collect above the cuff.

### SUMMARY OF THE INVENTION

The invention set forth herein is a tracheal tube having an inflatable cuff defining a proximal portion and a distal portion of the tube. The tracheal tube comprises a ventilation tube, a cuff inflation lumen, at least one suction lumen, a plurality of suction ports, and a plurality of recessed channels. The channels are disposed in a circumferential orientation on the exterior of the ventilation tube. Each suction lumen terminates in at least one suction port, each suction port opens onto a channel, and each channel is located on the proximal portion of the tube within less than 20 mm of the cuff.

Preferred embodiments have suctioning area above the cuff which features at least two suction ports, each opening into an associated channel, which is a circumferential groove extending partially around the exterior of the tube and subtending an angle of almost 180° around the tube. Each channel collects fluid from within a nearby volume of fluid, and directs the fluid to the associated suction port. Taken together, the channels subtend a region that is almost 360° around the tube. The walls of the channels serve to direct suctioning forces out from the suction ports and around the tube, and via capillary action, serve to direct fluids from around the tube into the ports. In addition, the channels and associated suction ports are disposed close to the inflatable cuff so as to minimize pooling of fluids below the channels. In certain embodiments, each individual suction port has an individual associated lumen to transport the suctioned fluids out of the body.

### BRIEF DESCRIPTION OF THE FIGURES

FIG. 1 shows a perspective view of a tracheostomy tube of the invention.

FIG. 2 is a partial view of the dorsal side of a tracheal tube of the invention.

FIG. 3 is a partial view of the left side of a tracheal tube of the invention.

FIG. 4 is a partial view of the right side a tracheal tube of the invention.

### DETAILED DESCRIPTION OF THE INVENTION

Embodiments of the present invention will now be described with reference to the above-identified drawings. However, the drawings and the descriptions herein are illustrative, and are not intended to limit the scope of the invention. Modifications of the examples of the invention described herein will be evident to those of skill in the art, and such modifications are within the scope and spirit of the invention. Also, features described herein may be omitted, additional features may be included, and/or features described herein may be combined in a manner different from the specific combinations recited herein, all without departing from the scope and spirit of the invention.

Intubation with a tracheal tube is performed by way of an incision in the patient's neck, or by inserting a flexible tube through a patient's oral (or nasal) cavity. By way of terminology, a tracheal tube that is inserted via the oral cavity is an endotracheal tube, whereas a tracheal tube inserted through a stoma in the neck is a tracheostomy tube. It will be understood that the invention relates to both endotracheal tubes and tracheostomy tubes. Accordingly, the terms “tracheal tube”, “endotracheal tube” and “tracheostomy tube” should be regarded as interchangeable for purposes of this disclosure, unless the particular context of the term requires otherwise. The inventive tracheal tube may be used with or without a ventilator (breathing machine). The angles subtended by the channels of the invention, as described below, do not have critical values; rather, the efficiency of the device will improve in a continuous manner as the subtended angles are increased. Any angle described as being “about” some value should thus be understood to describe and enable not only the stated value, but other angles within about 10° of the stated value. In general, the closer the total angle subtended is to 360°, the more preferable is the embodiment.

The tracheal tube of the invention is provided with an inflatable balloon or cuff, as is known in the art, which can be inflated to seal off the distal portion of the trachea, and the lungs, from the air in the subglottic region. The inflated cuff thereby enables the lungs to be inflated and deflated by a mechanical ventilator, and also helps to center the tracheal tube within the trachea. In the present invention, suction ports are provided above the cuff, arranged so that they suction fluids from the subglottic region when the device is deployed in a patient. In preferred embodiments of the invention, a mechanical aspirator (not shown) is used to create negative pressure in order to draw out fluids through the suction ports and deliver them to an external container.

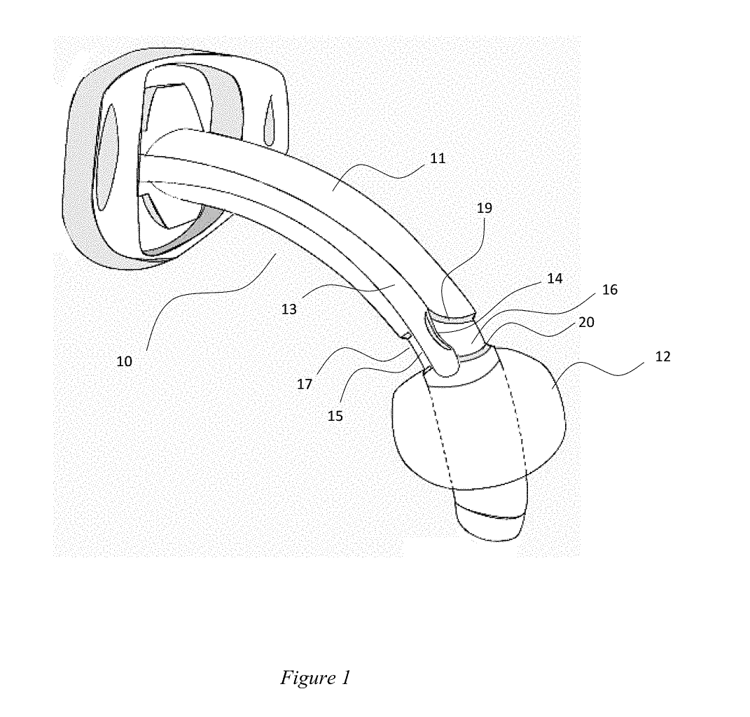

FIG. 1 is a perspective drawing of a tracheostomy tube 10 according to the invention. The main body of the device is an elongated, flexible ventilation tube 11 having a central lumen, which allows for the passage of air, which is typically provided by an external mechanical ventilator. The device is provided with a cuff or balloon 12 that, when inflated, seals the tube 10 against the inner wall of the trachea. A suction tube 13 is provided on the side of ventilation tube 11, which is externally connected to a vacuum source. The lumen of tube 13 is a suction lumen that carries away fluids through suction ports 14 and 15 (not visible in FIG. 1), which are located just above the cuff 12. The suction ports may be elongated, as shown in FIG. 1, but may be round, or may take any other shape that is convenient or desired. Suction port 14 opens out into a recessed channel 16 that is located just above the cuff 12 and which extends almost 180° around the circumference of the ventilation tube 11, subtending thereby an angle of almost 180° around the tube. Port 15 opens out onto a comparable recessed channel 17. The distance between the cuff and the channels 16 and 17 is less than 20 mm, and is as short as practicable, preferably less than 3 mm, more preferably less than 2 mm, and most preferably between 0 and 1 mm.

Channel 18 is defined along its length by walls 19 and 20, which are substantially orthogonal to the outer surface of ventilation tube 11 and to the floor of the channel. The channel is cut into the body of ventilation tube 11, preferably to a depth between about 0.5 mm and about 3 mm, consistent with the thickness of the walls of ventilation tube 11 and with due regard for the overall mechanical strength of the device 10. The width of the channels preferably ranges from about 1 mm to about 10 mm. The walls of ventilation tube 11 may optionally be thickened in the area of the channels, to compensate for the lack of material corresponding to the volume of the channels. The angle around the circumference of the tube that is subtended by each channel is at least about 160°, preferably at least 170°, and more preferably at least 175°. The sum of the angles subtended by both channels together is at least 320°, preferably at least 340°, more preferably at least 350°, and most preferably at least 355°. It is preferred that the channels do not merge so as to form a single channel, although such embodiments are contemplated, and may be employed if desired.

Prior art manufacturing techniques, including for example cutting, molding, adhering and welding, may be employed in making and assembling the tubes, lumens, and cuff, as is well known in the art. In certain embodiments, drilling, cutting and other machining methods will be preferable for creating ports and channels in close proximity to the cuff.

In the particular embodiment illustrated in FIG. 1, two suction ports are disposed approximately 180° apart on a single suction line which is external to ventilation tube 11. It will be appreciated that in alternative embodiments, the suction line may take the form of a lumen internal to, or within the walls of, ventilation tube 11. It will also be appreciated that two suction lumens may be employed, each of which opens out into a separate suction port; in these embodiments, the sucking of air through one non-submerged port will not reduce the effective vacuum at the other port, which may still be submerged in fluid. Multiple-lumen tracheal tubes are known in the art, and are disclosed, for example, in US patent publication No. 2016/0325063, the contents of which are incorporated herein by reference in their entirety.

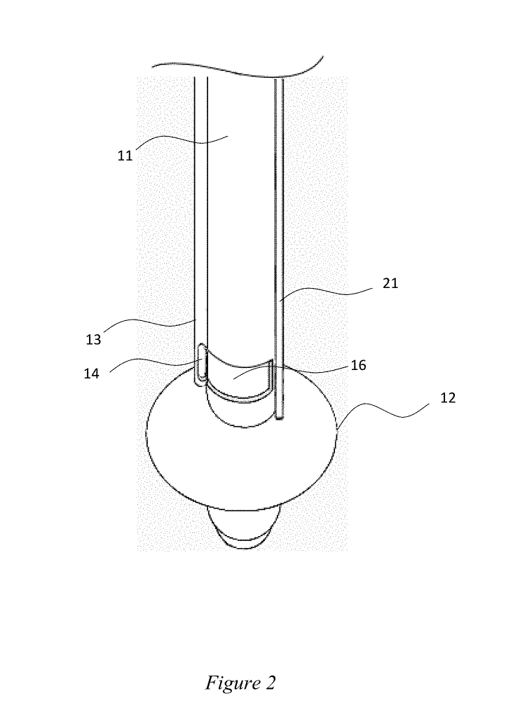

FIG. 2 shows a perspective, partial view of the dorsal side of a tracheal tube of the invention. As in prior art devices, a cuff inflation tube 21 extends from the exterior of the device to the cuff 11. The cuff inflation lumen of tube 21 carries air for inflating the cuff. In alternative embodiments, the cuff inflation lumen may take the form of a lumen internal to, or within the walls of, ventilation tube 11. Channel 16 extends from port 14 to the edge of tube 21, and will terminate at about this point even in those embodiments where the inflation lumen is internally located. Channel 17 (not visible in FIG. 2) is similarly disposed on the ventral side of ventilation tube 11, and is similarly dimensioned.

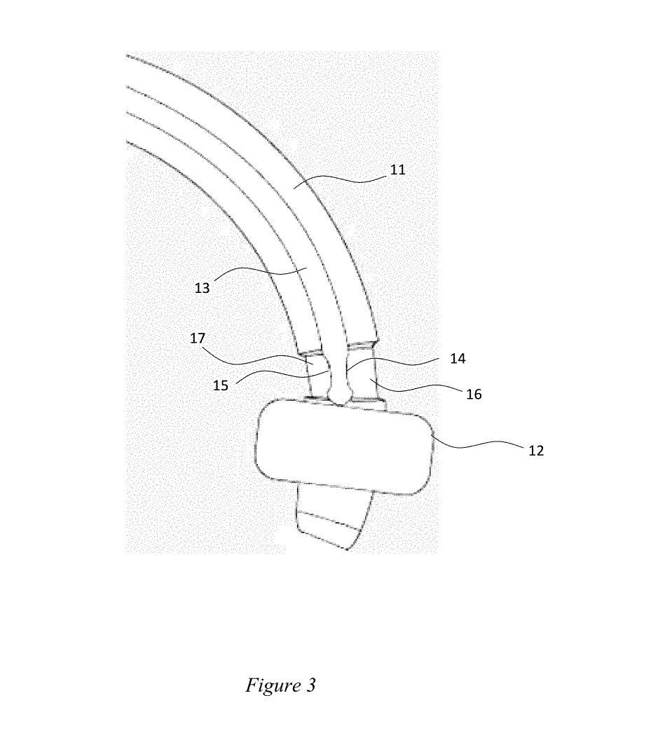

FIG. 3 shows a partial plane view of the right side of the tracheal device 10. In this embodiment, opposing sides of suction tube 13 are partially cut away just above the cuff 12 to form suction ports 14 and 15. The suction ports may be elongated, as shown in FIGS. 1-3, but they may optionally be round, or may take any other shape that is convenient or desired. It is preferred that the ports be dimensioned to approximate the width of channels 16 and 17. Suction port 14 opens out into channel 16, and port 15 opens out onto channel 17. As such, when suction is externally applied to suction tube 13, both suctions ports 14 and 15 are activated to aspirate fluids. Suction port 14 applies suction through channel 16, thereby aspirating the subglottal area around approximately 180° on the dorsal side of the tube, while suction port 15 applies suction through channel 17, thereby aspirating the subglottal area around approximately 180° on the ventral side of the tube.

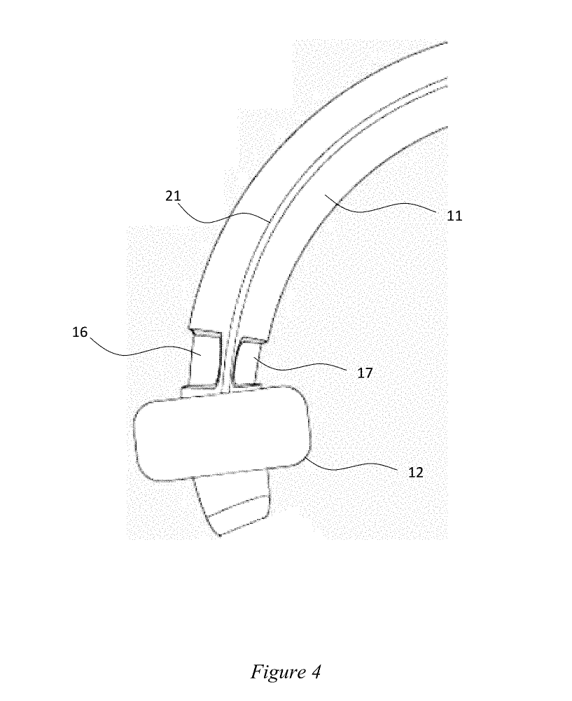

FIG. 4 shows a partial plane view of the left side of the tracheal device 10. In this view, the cuff inflation tube 21 is visible, with the two channels 16 and 17 terminating on either side. In certain embodiments, the inflation tube may be located within the ventilation tube 11, or may be replaced by a lumen within the walls of the ventilation tube 11. The two channels in such embodiments will preferably still terminate substantially as shown. In alternative embodiments, the two channels may be joined beneath the inflation tube 21, or, in embodiments where no external tube 21 is present, they may form a single contiguous channel spanning this area, so as to form a single channel subtending at least about 340° of the area around ventilation tube 11.

The various embodiments of the invention provide increased suctioning and prevent substantial pooling of fluids in patients having tracheostomies. Two suctioning ports are provided, each of which is capable of providing substantially 180° of vacuum coverage. Whereas prior systems could only suction from one localized suction port, the inventive tube provides roughly 360° of suctioning. Moreover, because the suctioning ports are within close proximity of the cuff, the ports are able to access low levels of fluid—thus, fluid levels need not be elevated before suctioning can be effective.

It will be understood by those of ordinary skill in the art that the approximately 180° of suction provided both ventrally and dorsally will allow a patient to be placed at any angle to facilitate optimal subglottic suctioning. Prior art subglottic suction tracheotomy tubes having a single dorsal suction port of a small size allow for only dorsal area suction with less than 90° coverage. The inventive tube described herein, on the other hand, allows for optimal subglottic suction at any patient angle and allows for suctioning of larger amounts of secretions due to its roughly 360° of coverage.

Having described this invention with regard to specific embodiments, it is to be understood that the description is not meant as a limitation since further modifications and variations may be apparent or may suggest themselves to those skilled in the art. It is intended that the present application cover all such modifications and variations.

I claim:

1. A tracheal tube having an inflatable cuff defining a proximal portion and a distal portion of the tube, wherein the tracheal tube comprises
a. a ventilation tube;
b. a cuff inflation lumen;
c. at least one suction lumen;
d. a plurality of suction ports; and
e. a plurality of recessed channels, disposed in a circumferential orientation on the exterior of the ventilation tube;
wherein each suction lumen terminates in at least one suction port, each suction port opens onto a channel, and each channel is located on the proximal portion of the tube within less than 20 mm of the cuff.

2. The tracheal tube according to claim 1, wherein there are two suction ports and two channels, each channel subtending at least 160° of the circumference of the ventilation tube.

3. The tracheal tube according to claim 2, wherein there are two suction lumens, each suction lumen terminating in a single suction port.

4. The tracheal tube according to claim 2, wherein there is single suction lumen, the suction lumen terminating in two suction ports.

5. The tracheal tube according to claim 1, wherein each channel is located on the proximal portion of the tube within less than 10 mm of the cuff.

6. The tracheal tube according to claim 1, wherein each channel is located on the proximal portion of the tube within less than 5 mm of the cuff.

7. The tracheal tube according to claim 1, wherein each channel is located on the proximal portion of the tube within less than 2 mm of the cuff.

8. The tracheal tube according to claim 2, wherein each channel is located on the proximal portion of the tube within less than 10 mm of the cuff.

9. The tracheal tube according to claim 2, wherein each channel is located on the proximal portion of the tube within less than 5 mm of the cuff.

10. The tracheal tube according to claim 2, wherein each channel is located on the proximal portion of the tube within less than 2 mm of the cuff.

11. The tracheal tube according to claim 3, wherein each channel is located on the proximal portion of the tube within less than 10 mm of the cuff.

12. The tracheal tube according to claim 3, wherein each channel is located on the proximal portion of the tube within less than 5 mm of the cuff.

13. The tracheal tube according to claim 3, wherein each channel is located on the proximal portion of the tube within less than 2 mm of the cuff.

14. The tracheal tube according to claim 4, wherein each channel is located on the proximal portion of the tube within less than 10 mm of the cuff.

15. The tracheal tube according to claim 4, wherein each channel is located on the proximal portion of the tube within less than 5 mm of the cuff.

16. The tracheal tube according to claim 4, wherein each channel is located on the proximal portion of the tube within less than 2 mm of the cuff.

17. The tracheal tube according to claim 2, wherein there are two suction ports and two channels, each channel subtending at least 170° of the circumference of the ventilation tube.

18. The tracheal tube according to claim 3, wherein there are two suction ports and two channels, each channel subtending at least 170° of the circumference of the ventilation tube.

19. The tracheal tube according to claim 4, wherein there are two suction ports and two channels, each channel subtending at least 170° of the circumference of the ventilation tube.

20. The tracheal tube according to claim 16, wherein there are two suction ports and two channels, each channel subtending at least 170° of the circumference of the ventilation tube.
